(19) United States
(12) Patent Application Publication
(10) Pub. No.: US 2018/0222827 A1
(43) Pub. Date: Aug. 9, 2018

(54) DICHLOROMETHANE REDUCTION FROM A METHANE OXYCHLORINATION PRODUCT STREAM

(71) Applicant: SABIC Global Technologies B.V., Bergen op Zoom (NL)

(72) Inventors: Kaiwalya SABNIS, Sugar Land, TX (US); Dustin FICKEL, Sugar Land, TX (US); Heng SHOU, Sugar Land, TX (US); Edouard MAMEDOV, Sugar Land, TX (US)

(73) Assignee: SABIC Global Technologies B.V.

(21) Appl. No.: 15/944,890

(22) Filed: Apr. 4, 2018

Related U.S. Application Data
(60) Provisional application No. 62/562,692, filed on Sep. 25, 2017.

(51) Int. Cl. C07C 17/154; C07C 19/03; B01J 27/10; B01J 29/06; B01J 29/85

(52) U.S. Cl. CPC C07C 17/154; C07C 19/03; B01J 27/10; C07C 2/84; B01J 29/85; B01J 2231/46; B01J 29/06

(57) ABSTRACT

A chemical reactor system includes: a feed; a methane oxychlorination catalyst, wherein a product of an oxychlorination reaction is dichloromethane; and a dichloromethane conversion catalyst, wherein the dichloromethane conversion catalyst provides a product stream having a dichloromethane selectivity less than 5%. The addition of the dichloromethane conversion catalyst to the reactor bed can decrease the amount of dichloromethane produced and increase the amount of monochloromethane produced. Accordingly, dichloromethane does not have to be separated from the product stream and the monochloromethane can then be used to produce other products, such as olefins.

### TECHNICAL FIELD

Methane oxychlorination followed by conversion of methyl chloride (monochloromethane) can be used to convert chloromethane into valuable light olefins, such as ethylene and propylene, and silicone polymers. Methane oxychlorination also produces dichloromethane (DCM), which cannot be directly converted to light olefins. A dichloromethane conversion catalyst can be used to convert DCM into chloromethane, trichloromethane, and other products.

### BRIEF DESCRIPTION OF THE FIGURES

The features and advantages of certain embodiments will be more readily appreciated when considered in conjunction with the accompanying figures. The figures are not to be construed as limiting any of the preferred embodiments.

FIG. 1 is an illustration of a reactor bed containing interspersed methane oxychlorination and dichloromethane conversion catalysts according to certain embodiments.

FIG. 2 is an illustration of a reactor bed containing layered methane oxychlorination and dichloromethane conversion catalysts according to certain other embodiments.

### DETAILED DESCRIPTION OF THE INVENTION

Methane oxychlorination followed by conversion of methyl chloride (monochloromethane) to olefins is one of the possible routes to convert methane into valuable light olefins, such as ethylene and propylene. Some light olefins are used as intermediaries to produce other industrial products. For example, ethylene is one of the largest organic chemical feedstocks by volume that can be used to produce polymers, such as polyethylene, and many other chemicals and products.

Oxychlorination of methane is a process to synthesize methyl chloride by reacting methane with hydrogen chloride and oxygen in presence of a methane oxychlorination catalyst in a fixed-bed reactor. This process also produces dichloromethane (DCM) as a byproduct. Unlike methyl chloride (also known as monochloromethane or chloromethane), DCM cannot be directly converted to light olefins.

Currently, the process used to address the unusable formation of dichloromethane is to separate the dichloromethane from the oxychlorination product stream to obtain pure chloromethane, wherein the pure chloromethane can then be sent to an olefin synthesis reactor. In short, formation of dichloromethane is widely regarded as an inevitable product during the methane oxychlorination reaction that has to be separated. Dichloromethane products also result in an overall carbon less from the system; thus, reducing the amount of olefins that may be produced. One way to reduce or avoid the formation of dichloromethane is by operating the methane oxychlorination reactor at a low methane conversion. However, the result is a low overall productivity of the useful chloromethane product. Thus, there is a need and an on-going industry wide concern to reduce or eliminate the amount of dichloromethane produced during methane oxychlorination without negatively impacting the amount of chloromethane produced.

It has been discovered that dichloromethane can be significantly reduced or eliminated from the methane oxychlorination product stream before the product stream exits the reactor. A second catalyst can be included in the reactor bed. The second catalyst is a dichloromethane conversion catalyst, wherein a given number of moles of dichloromethane are converted into an equimolar mixture of chloromethane and trichloromethane (chloroform). In presence of water that is formed in the methane oxychlorination reaction, the trichloromethane can be further converted into carbon monoxide and hydrogen chloride. Thus, the product stream from the reactor can include only products that are useful and very little to no separation of dichloromethane needs to be performed. Moreover, the dual catalyst system decreases the carbon losses from target olefins in a methane to olefins system and allows the oxychlorination reactions to be pushed towards higher conversions of useful products, such as monochloromethane.

According to certain embodiments, a chemical reactor system comprises: a feed; a methane oxychlorination catalyst, wherein a product of an oxychlorination reaction is dichloromethane; and a dichloromethane conversion catalyst, wherein the dichloromethane conversion catalyst provides a product having a dichloromethane selectivity less than 5%.

According to certain other embodiments, a method of reducing the amount of dichloromethane in a product stream comprises: introducing a feed into a reactor, wherein the reactor comprises: a methane oxychlorination catalyst; and a dichloromethane conversion catalyst; allowing the feed to chemically react with the methane oxychlorination catalyst, wherein a product of the methane oxychlorination chemical reaction is dichloromethane; and allowing the dichloromethane to chemically react with the dichloromethane conversion catalyst, wherein the product from the dichloromethane conversion reaction has a dichloromethane selectivity less than 5%.

It is to be understood that any discussion of the various embodiments regarding the reactor and catalysts are intended to apply to the system and method embodiments.

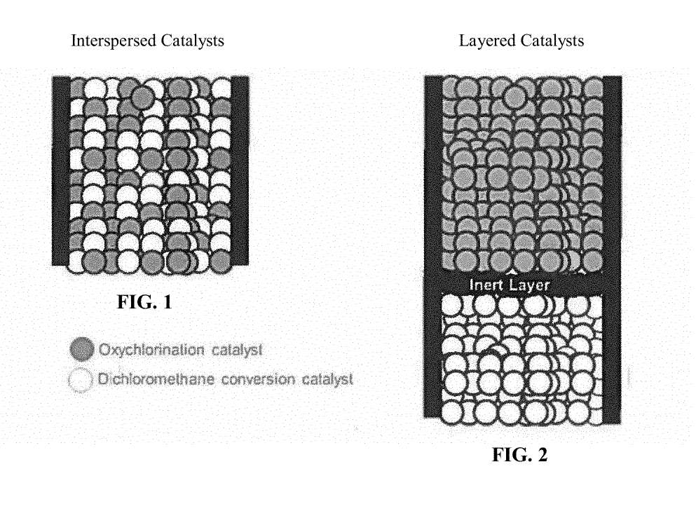

Turning to the Figures, FIG. 1 shows a reactor bed according to certain embodiments. The system contains a reactor bed. The reactor bed can be any type of reactor, such as a fixed-bed reactor. The reactor can be used for a methane oxychlorination reaction. The reactor bed can include a methane oxychlorination catalyst and a dichloromethane conversion catalyst. As shown in FIG. 1, the methane oxychlorination catalyst and the dichloromethane conversion catalyst can be interspersed with each other in the reactor bed.

FIG. 2, shows a reactor bed according to certain other embodiments. According to these other embodiments, the reactor bed can include a layer of the methane oxychlorination catalyst and a separate layer of the dichloromethane conversion catalyst. The dichloromethane conversion catalyst can be located downstream of the methane oxychlorination catalyst within the reactor bed. As used herein, the term “downstream” means at a location away from the feed entry point into the reactor bed. Accordingly, a downstream catalyst layer would receive reaction products of the feed and/or unreacted components of the feed. The reactor bed can further include an inert layer of material located between the methane oxychlorination catalyst and the dichloromethane conversion catalyst. The inert layer preferably does not chemically react with the feed or any reaction products from the catalysts. The inert layer of material can be selected from the group consisting of quartz wool, quartz chips, silicon carbide, silica wool, ceramic packing, an empty void, or combinations thereof.

The thickness of the layers of the methane oxychlorination catalyst, the dichloromethane conversion catalyst, and the inert layer can vary and can be selected wherein the dichloromethane conversion catalyst provides products having a dichloromethane selectivity less than 5%, preferably, less than 1%. According to certain embodiments, the thickness of the methane oxychlorination catalyst and the dichloromethane conversion catalyst are the same. The thickness of the methane oxychlorination catalyst and the dichloromethane conversion catalyst can vary and be selected to provide a product from the dichloromethane conversion reaction having a dichloromethane selectivity less than 5%.

The system includes a feed and the methods include introducing the feed into a reactor. The feed includes methane, hydrogen chloride, and a source of oxygen. The source of oxygen can include, but is not limited to, air, pure oxygen (e.g., dioxygen), or nitrous oxide (e.g., dinitrogen monoxide or nitric oxide).

The feed can come in contact with the methane oxychlorination catalyst. The methane oxychlorination catalyst can be any catalyst that causes oxychlorination of methane. The methane oxychlorination catalyst can be, for example, selected from the group consisting of metal oxides (e.g., lanthanum oxide, cerium(IV) oxide, and iron(III) oxide), mixed metal oxides (e.g., lanthanum oxide-cerium oxide, iron oxide-cerium oxide), and supported metal chlorides.

The products from the oxychlorination of methane reaction can include chloromethane, dichloromethane, trichloromethane, carbon tetrachloride, carbon monoxide, carbon dioxide, and water.

The reactor system also includes a dichloromethane conversion catalyst. In order to convert dichloromethane into products, the dichloromethane conversion catalyst includes a material possessing hydroxyl functional groups. Examples of materials possessing hydroxyl functional groups include, but are not limited to, compounds selected from the group consisting of metal oxides from Group 2-7 and 12-15 of the periodic table (e.g., zirconium oxide, zinc oxide, aluminum oxide, and gallium oxide), non-metal or metalloid oxides from Group 13-15 of the periodic table (e.g., silicon oxide), aluminosilicate zeolites (e.g., ZSM-5, zeolite beta, and SSZ-13), silicoaluminophosphates (e.g., SAPO-34, SAPO-5, SAPO-11, and SAPO-18), mixed oxides selected from Group 2-7 and 12-15 elements of the periodic table (e.g., silica-alumina oxide, magnesium-aluminum oxide, hydrotalcite, and titanium-zirconium oxide), and combinations thereof.

During the dichloromethane conversion reaction, the products from the oxychlorination reaction react with the dichloromethane conversion catalyst to convert dichloromethane into other products. The products of the dichloromethane conversion reaction can include, without limitation, carbon monoxide, carbon dioxide, and monochloromethane (i.e., chloromethane). An example chemical reaction scheme can include that for every 2 moles of dichloromethane, there are 1 mole each of monochloromethane and trichloromethane produced. The trichloromethane can then react with the hydroxyl groups to produce carbon monoxide, and hydrochloric acid. As the reaction proceeds, available hydroxyl groups may become depleted. Accordingly, water from the oxychlorination reaction can replenish/regenerate the depleted hydroxyl groups on the dichloromethane conversion catalyst.

A higher concentration of unconverted oxygen from the oxychlorination reaction can drive the dichloromethane conversion reaction to produce more carbon monoxide and carbon dioxide instead of the desired product of monochloromethane. As such, it is desirable for a high oxygen conversion to occur during the oxychlorination reaction. According to certain embodiments, the oxychlorination reaction provides an oxygen conversion from the source of oxygen that is greater than or equal to 90%, preferably equal to 100%. The methane oxychlorination catalyst can be selected to provide an oxygen conversion from the source of oxygen that is greater than or equal to 90%, preferably equal to 100%. The concentrations of the methane oxychlorination catalyst and oxygen can also be selected to provide an oxygen conversion from the source of oxygen that is greater than or equal to 90%, preferably equal to 100%. As the reactions in the reactor bed proceed, it may be necessary to replenish or add more of the methane oxychlorination catalyst in order to achieve the desired amount of oxygen conversion.

According to certain embodiments, the products of the dichloromethane conversion catalyst have greater monochloromethane content than the product of the oxychlorination catalyst. In other words, the amount of monochloromethane in the mixture exiting the reactor is higher than the amount of monochloromethane that would exit if only a methane oxychlorination catalyst were present in the reactor. Accordingly, the addition of the dichloromethane conversion catalyst not only decreases the amount of dichloromethane produced, but can also increase the amount of monochloromethane.

The particle size of the dichloromethane conversion catalyst can vary and can be selected to provide products having a dichloromethane selectivity less than 5%, preferably, less than 1%. By way of example, a smaller particle size will increase the surface area of the dichloromethane conversion catalyst wherein more hydroxyl groups are available to react with the dichloromethane. According to certain embodiments, the particle size of the dichloromethane conversion catalyst is in the range from about 20 to about 40 mesh.

The concentration of the dichloromethane conversion catalyst can vary and can be selected to provide products having a dichloromethane selectivity less than 5%, preferably, less than 1%. By way of example, a smaller particle size will increase the surface area of the dichloromethane conversion catalyst wherein more hydroxyl groups are available to react with the dichloromethane. According to certain embodiments, the concentration of the dichloromethane conversion catalyst is in the range from about 1% to about 200% by weight of the methane oxychlorination catalyst.

The reactor conditions can vary and be selected to provide products having a dichloromethane selectivity less than 5%, preferably, less than 1%. One of ordinary skill in the art will be able to select the appropriate operating conditions to provide the desired selectivity of products. According to certain embodiments, the reactor is operated at a temperature in the range from about 350° C. to about 500° C., a pressure in the range from about 1 bar to about 15 bar, and a weight hourly space velocity in the range from about 0.1/hr to about 10/hr.

The methods include introducing a feed into a reactor, wherein the reactor comprises: a methane oxychlorination catalyst; and a dichloromethane conversion catalyst; allowing the feed to chemically react with the methane oxychlorination catalyst, wherein a product of the methane oxychlorination chemical reaction is dichloromethane; and allowing the dichloromethane to chemically react with the dichloromethane conversion catalyst, wherein the product from the dichloromethane conversion reaction has a dichloromethane selectivity less than 5%.

The methods can further include using the products from the dichloromethane conversion reaction to produce other products. By way of example, the methods can further include feeding the monochloromethane product into a reactor to produce olefins. Examples of produced olefins can include, without limitation, ethylene, propylene, and intermediates in silicone polymer production.

### Examples

To facilitate a better understanding of the present invention, the following examples of certain aspects of preferred embodiments are given. The following examples are not the only examples that could be given according to the present invention and are not intended to limit the scope of the invention.

The results in the following Table were obtained by feeding a mixture of methane, hydrogen chloride, and a source of oxygen comprising 20% methane, 20% hydrogen chloride, 8% oxygen, and 52% nitrogen into a fixed-bed reactor containing a methane oxychlorination (abbreviated as M.O.) catalyst alone or a layered catalyst bed containing the methane oxychlorination catalyst and a dichloromethane (abbreviated as D.C.M.) conversion catalyst. The methane oxychlorination catalyst was cerium oxide and the dichloromethane conversion catalyst was either a silicoaluminophosphates of SAPO-34 or a mixed oxide of gamma aluminum oxide. The reactor bed was operated at a temperature of 450° C. and a weight hourly space velocity of 1/hr. The weight ratio of the dichloromethane conversion catalyst to methane oxychlorination catalyst was 1:3.33. The inert layer located between the methane oxychlorination catalyst and the dichloromethane conversion catalyst in the reactor bed was quartz wool.

TABLE 1

| | | M.O. catalyst + D.C.M. | M.O. catalyst + D.C.M. |
|---|---|---|---|
| | | conversion catalyst of | conversion catalyst of |
| | M.O. catalyst only | SAPO-34 | γ-Al₂O₃ |
| Methane Conversion (%) | 40.1 | 39.4 | 42.8 |
| Chloromethane (CH₃Cl) Selectivity (%) | 54.3 | 61.0 | 63.2 |
| Dichloromethane (CH₂Cl₂) Selectivity(%) | 19.9 | 0.0 | 0.7 |
| Trichloromethane (CHCl₃) Selectivity (%) | 0.7 | 0.2 | 0.0 |
| Carbon Tetrachloride (CCl₄) Selectivity (%) | — | 0.1 | 0.1 |
| Carbon Monoxide (CO) Selectivity (%) | 20.8 | 32.9 | 30.8 |
| Carbon Dioxide (C0₂) Selectivity (%) | 4.3 | 5.8 | 5.2 |

As can be seen in Table 1, the addition of a second layer of a dichloromethane conversion catalyst, reduces the selectivity of the undesirable product dichloromethane to less than 1%, while simultaneously increasing the selectivity of desired products of chloromethane and carbon monoxide. These results show that by adding a dichloromethane conversion catalyst to the reactor bed, separation of dichloromethane (DCM) is eliminated because the conversion of DCM is achieved. This shows an economical and efficient way to convert dichloromethane into useful product simultaneously with a methane oxychlorination reaction.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is, therefore, evident that the particular illustrative embodiments disclosed above may be altered or modified and all such variations are considered within the scope and spirit of the present invention.

As used herein, the words “comprise,” “have,” “include,” and all grammatical variations thereof are each intended to have an open, non-limiting meaning that does not exclude additional elements or steps. While compositions, systems, and methods are described in terms of “comprising,” “containing,” or “including” various components or steps, the compositions, systems, and methods also can “consist essentially of” or “consist of” the various components and steps. It should also be understood that, as used herein, “first,” “second,” and “third,” are assigned arbitrarily and are merely intended to differentiate between two or more phases, etc., as the case may be, and does not indicate any sequence. Furthermore, it is to be understood that the mere use of the word “first” does not require that there be any “second,” and the mere use of the word “second” does not require that there be any “third,” etc.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent(s) or other documents that may be incorporated herein by reference, the definitions that are consistent with this specification should be adopted.

What is claimed is:

1. A chemical reactor system comprising:
a feed;
a methane oxychlorination catalyst, wherein a product of an oxychlorination reaction is dichloromethane; and
a dichloromethane conversion catalyst, wherein the dichloromethane conversion catalyst provides a product having a dichloromethane selectivity less than 5%.

2. The system according to claim 1, wherein the methane oxychlorination catalyst and the dichloromethane conversion catalyst are interspersed within a reactor bed.

3. The system according to claim 1, wherein the dichloromethane conversion catalyst is located downstream of the methane oxychlorination catalyst within a reactor bed.

4. The system according to claim 3, further comprising an inert layer of material located between the methane oxychlorination catalyst and the dichloromethane conversion catalyst.

5. The system according to claim 4, wherein the inert layer of material comprises quartz wool, quartz chips, silicon carbide, silica wool, ceramic packing, an empty void, or combinations thereof.

6. The system according to claim 1, wherein the feed comprises methane, hydrogen chloride, and a source of oxygen.

7. The system according to claim 1, wherein the methane oxychlorination catalyst is selected from the group consisting of metal oxides, mixed metal oxides, and supported metal chlorides.

8. The system according to claim 1, wherein the product from the oxychlorination reaction comprises at least one of chloromethane, dichloromethane, trichloromethane, carbon tetrachloride, carbon monoxide, carbon dioxide, and water.

9. The system according to claim 1, wherein the oxychlorination reaction provides an oxygen conversion greater than or equal to 90%.

10. The system according to claim 9, wherein the concentration of the methane oxychlorination catalyst and oxygen are selected to provide an oxygen conversion greater than or equal to 90%.

11. The system according to claim 1, wherein the dichloromethane conversion catalyst comprises a material possessing hydroxyl functional groups.

12. The system according to claim 11, wherein the dichloromethane conversion catalyst is selected from the group consisting of metal oxides from Groups 2-7 and 12-15 of the periodic table, non-metal or metalloid oxides from Groups 13-15 of the periodic table, aluminosilicate zeolites, silicoaluminophosphates, mixed oxides selected from Groups 2-7 and 12-15 elements of the periodic table, and combinations thereof.

13. The system according to claim 1, wherein products of a dichloromethane conversion reaction comprise carbon monoxide, carbon dioxide, and monochloromethane.

14. The system according to claim 1, wherein the concentration of the dichloromethane conversion catalyst is in the range from about 1% to about 200% by weight of the methane oxychlorination catalyst.

15. The system according to claim 1, wherein the reactor is operated at a temperature in the range from about 350° C. to about 500° C.

16. The system according to claim 1, wherein the reactor is operated at a pressure in the range from about 1 bar to about 15 bar.

17. A method of reducing the amount of dichloromethane in a product stream comprising:
introducing a feed into a reactor, wherein the reactor comprises:
a methane oxychlorination catalyst; and
a dichloromethane conversion catalyst;
allowing the feed to chemically react with the methane oxychlorination catalyst, wherein a product of the methane oxychlorination chemical reaction is dichloromethane; and
allowing the dichloromethane to chemically react with the dichloromethane conversion catalyst, wherein the product from the dichloromethane conversion reaction has a dichloromethane selectivity less than 5%.

18. The method according to claim 17, further comprising feeding the monochloromethane into a reactor to produce olefins and intermediates in silicone polymer production.

19. A dual catalyst system comprising:
a methane oxychlorination catalyst; and
a dichloromethane conversion catalyst,
wherein the product of the dichloromethane conversion reaction has a greater monochloromethane content than the product of the methane oxychlorination reaction, and
wherein the dual catalysts decrease carbon losses from target olefins in a methane to olefins system, and push oxychlorination reactions towards higher conversions of monochloromethane.

20. The system according to claim 19, wherein the dichloromethane conversion catalyst provides a product having a dichloromethane selectivity less than 5%.
